(19) United States
(12) Patent Application Publication
(10) Pub. No.: US 2026/0274777 A1
(43) Pub. Date: Sep. 17, 2026

(54) METHOD FOR PRODUCING DIMETHYLETHER

(71) Applicant: Fraunhofer-Gesellschaft zur Förderung der angewandten Forschung e.V., München (DE)

(72) Inventors: Malte Gierse, Freiburg (DE); Benedikt Steinbach, Freiburg (DE); Mohamed Ouda, Freiburg (DE)

(21) Appl. No.: 18/873,819

(22) PCT Filed: May 31, 2023
(86) PCT No.: PCT/EP2023/064558

§ 371 Date: Dec. 11, 2024

(30) Foreign Application Priority Data
Jun. 13, 2022 (DE) 10 2022 114 811.4

(51) Int. Cl. C07C 41/09

(52) U.S. Cl. CPC C07C 41/09

(57) ABSTRACT

The present invention relates to a process for the preparation of dimethyl ether comprising the following steps:
feeding a methanol-containing stream S_{Feed-RD} into a reactive distillation unit RD, wherein
   in at least one reaction zone RZ of the reactive distillation unit RD, methanol is converted to dimethyl ether and water in the presence of an acid catalyst, and
   a distillative separation is performed into a fraction that contains dimethyl ether and leaves the reactive distillation unit RD as a overhead stream S_{overhead-RD}, and a fraction that contains water and leaves the reactive distillation unit RD as a bottom stream S_{bottom-RD},
removing a methanol-containing side stream S_{side-RD} from the reactive distillation unit RD,
feeding the methanol-containing side stream S_{side-RD} into a side reactor SR and converting the methanol in the presence of an acid catalyst to dimethyl ether and water to obtain a product stream S_{product-SR}, which contains dimethyl ether, water and methanol and is removed from the side reactor SR.

Dimethyl ether is an industrially important starting material for the preparation of dimethyl sulfate and is used as a propellant and refrigerant. Dimethyl ether is also of interest as a synthetic fuel, for example as a substitute for LPG and diesel fuel.

An overview of known processes for the preparation of dimethyl ether can be found in the following publications:
Z. Azizi et al, “Dimethyl ether: A review of technologies and product challenges”, Chemical Engineering and Processing, 2014, 82, pp. 150-172;
V. Dieterich et al, “Power-to-liquid via synthesis of methanol, DME or Fischer-Tropsch fuels: a review”, Energy Environ. Sci., 2020, 13, pp. 3207-3252;
Th. Cholewa et al, “Process Intensification Strategies for Power-to-X Technologies”, ChemEngineering, 2022, 6(1), 13.

On a large scale, dimethyl ether (DME) can be produced in a reactor (e.g. a fixed-bed reactor) by dehydrating methanol in the presence of an acid catalyst. The dehydration reaction can be represented by the following reaction equation:

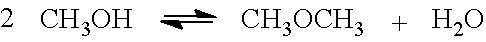

Gaseous methanol is usually fed into the DME synthesis reactor and converted to dimethyl ether and water at a reaction temperature of around 220-400° C. The dehydration reaction taking place in the DME synthesis reactor is thermodynamically limited (equilibrium-limited reaction) and usually yields a conversion of no more than around 70-85%.

The DME synthesis reactor is usually followed by at least two distillation steps, wherein dimethyl ether is separated from methanol and water in the first distillation step and methanol is separated from water in the second distillation step. The methanol separated by distillation is returned to the DME synthesis reactor.

The methanol fed to the DME synthesis reactor can be produced from synthesis gas in a known manner. The methanol obtained directly in this synthesis is also referred to as crude methanol and usually contains significant amounts of water (e.g. 20-50 mol %), especially in the case of sustainable methanol synthesis using a CO₂-rich and regenerative hydrogen-containing synthesis gas. A DME synthesis process in which the raw methanol can be used directly (i.e. without further processing such as water separation) as a starting material would therefore be of interest.

If the water-containing crude methanol is added to the DME synthesis reactor as a reactant stream, this has a detrimental effect on the conversion, as the equilibrium of the dehydration reaction is shifted in the direction of the reactant (methanol). In addition, some acidic solid catalysts such as γ-Al2O3 can be impaired in their catalytic activity and stability by the presence of significant amounts of water. Therefore, it is common practice to feed a substantially anhydrous methanol as a reactant stream to the DME synthesis reactor.

The conventional process for the preparation of dimethyl ether is therefore disadvantageous both in terms of its energy balance and the equipment required, since
the water from the raw methanol must be separated to obtain substantially anhydrous methanol, which can then be fed into the DME synthesis reactor,
the methanol must be vaporised before it is fed into the DME synthesis reactor, and
at least two distillation stages are connected downstream of the DME synthesis reactor, in which dimethyl ether is first separated from water and methanol and then methanol is separated from water by distillation.

If the conversion of methanol to dimethyl ether and water does not take place in the gas phase but in the liquid phase, the DME synthesis reactor can in principle be operated isothermally, e.g. at a temperature optimised for catalytic activity. A fixed bed reactor might be used for such isothermal operation. However, even with isothermal operation of the DME synthesis reactor, the hydration reaction of methanol to dimethyl ether and water is thermodynamically limited, so that a conversion of more than 85% cannot usually be realised.

It is known that the amount of equipment required in dimethyl ether synthesis can be reduced by using a reactive distillation unit. A reactive distillation unit (e.g. in the form of a reactive distillation column) contains one or more reaction zones in which reactants are reacted with one another, usually in the presence of a catalyst immobilised in the reaction zone, and one or more distillative separation zones in which reaction products and, if present, unreacted reactants are separated from one another.

US 2007/0066855 A1 describes a process for the preparation of dimethyl ether in which a methanol-containing stream is introduced into a reactive distillation column, methanol is converted into dimethyl ether and water in a reaction zone of the reactive distillation column in the presence of an acid catalyst and a distillative separation into a head stream, which substantially consists of dimethyl ether, and a sump stream, which substantially consists of water, takes place.

There is usually an optimum reaction temperature for a catalyst with regard to the reaction to be catalysed, e.g. high enough for sufficient catalytic activity, but not too high to avoid thermal degradation of the catalyst material or to minimise undesirable side reactions. In principle, a chemical reactor can be operated isothermally (e.g. at a temperature optimised for catalytic activity). However, when operating a reactive distillation unit, the temperature in the reaction zone usually decreases towards the top of the column. Therefore, the optimum reaction temperature can only be realised in part of the reaction zone. As a result, the reaction zone has areas with decreasing catalytic activity. The average catalytic activity of a specific catalyst that can be realised in the reaction zone of a reactive distillation unit is more or less clearly below the maximum catalytic activity that can be achieved with this catalyst.

To compensate for this, the volume of the reaction zone can be increased, for example. This requires the insertion of additional internals or packings with immobilised catalyst into the reaction zone, which is complex and costly.

Z. Lei et al, “Synthesis of dimethyl ether (DME) by catalytic distillation”, Chemical Engineering Science, 2011, 66, pp. 3195-3203, describe, among other things, a process (referred to as “Process A” in the publication) in which (i) the methanol-containing stream is first fed into a DME synthesis reactor to convert methanol to dimethyl ether and water in the presence of an acid catalyst and (ii) the product stream (containing dimethyl ether, water and unreacted methanol) removed from the DME synthesis reactor is fed directly into a reactive distillation unit to convert the remaining methanol to dimethyl ether and water and to perform a distillative separation into an overhead stream consisting substantially of dimethyl ether and a bottom stream consisting substantially of water. Since a DME synthesis reactor is connected upstream of the reactive distillation unit and a large proportion of the methanol has already been converted to dimethyl ether in this upstream DME synthesis reactor, the stream fed to the reactive distillation unit contains the desired end product (dimethyl ether) in a relatively high concentration. Therefore, only the methanol not converted in the DME synthesis reactor needs to be converted in the reactive distillation unit. The presence of the upstream DME synthesis reactor means that the reactive distillation unit can be smaller.

The “Process A” described in the publication by Z. Lei et al. (i.e. using an upstream DME synthesis reactor of which the product stream is fed into a reactive distillation unit) is disadvantageous, however, if crude methanol, i.e. methanol with a significant proportion of water, is fed to the DME synthesis reactor. The presence of water in the reactant stream inhibits the conversion of the dehydration reaction of methanol to dimethyl ether and water in the DME synthesis reactor. To counteract this loss of conversion, the reaction temperature in the DME synthesis reactor or the volume of the catalyst-containing reaction zone in the DME synthesis reactor could be increased. However, these measures increase the energy input of the process. In addition, an increase in the reaction temperature requires that the acid catalyst used for the dehydration reaction of the methanol has a sufficiently high thermal stability.

A problem addressed by the present invention is the preparation of dimethyl ether by an efficient (e.g. energy-efficient) process. In particular, the process should enable efficient preparation of dimethyl ether if crude methanol (i.e. methanol with a significant proportion of water) is used as the starting material. As already mentioned above, a raw methanol with a high water content is produced in particular during sustainable methanol synthesis using a CO₂-rich synthesis gas containing regenerative hydrogen.

The problem is solved by a process for the preparation of dimethyl ether, which comprises the following steps:
feeding a methanol-containing stream S_{Feed-RD} into a reactive distillation unit RD, wherein
   in at least one reaction zone RZ of the reactive distillation unit RD, methanol is converted to dimethyl ether and water in the presence of an acid catalyst, and
   a distillative separation is performed into a fraction that contains dimethyl ether and leaves the reactive distillation unit RD as an overhead stream S_{overhead-RD}, and a fraction that contains water and leaves the reactive distillation unit RD as a bottom stream S_{bottom-RD},
removing a methanol-containing side stream S_{side-RD} from the reactive distillation unit RD,
feeding the methanol-containing side stream S_{side-RD} into a side reactor SR and reacting the methanol in the presence of an acid catalyst to form dimethyl ether and water to obtain a product stream S_{product-SR}, which contains dimethyl ether, water and methanol and which is removed from the side reactor SR.

The process according to the invention is particularly suitable for the use of crude methanol (i.e. methanol which has a significant water content) as the feed stream.

During operation of the reactive distillation unit RD fed with crude methanol, substantially the very volatile dimethyl ether is removed via the overhead stream S_{overhead-RD} and substantially the high-boiling water is removed via the bottom stream S_{bottom-RD}, while a side stream S_{side-RD} can be discharged at a suitable point or height of the reactive distillation unit RD, which has a significantly higher methanol concentration or a significantly lower water concentration compared to the crude methanol. This side stream S_{side-RD} removed from the reactive distillation unit RD enables a very efficient conversion of the methanol to dimethyl ether in the side reactor SR, which functions as a DME synthesis reactor, due to the lower water concentration compared to the crude methanol. This allows a product stream S_{product-SR} with a high DME concentration to be produced in the side reactor SR, which also has a favourable influence on the yield of dimethyl ether in the reactive distillation unit RD, for example, after it is returned to the reactive distillation unit RD due to the high dimethyl ether concentration.

In the context of the present invention, the reactive distillation unit RD is thus used not only for the synthesis and distillative separation of the dimethyl ether, but also for the provision of a methanol source which has a lower water concentration compared to the crude methanol and thus enables a higher DME yield (compared to crude methanol) in a DME synthesis reactor.

As mentioned above, in the process according to the invention, a methanol-containing stream S_{Feed-RD} is fed into a reactive distillation unit RD, wherein
in at least one reaction zone RZ of the reactive distillation unit RD, methanol is converted to dimethyl ether and water in the presence of an acid catalyst, and
a distillative separation into a fraction containing dimethyl ether, which leaves the reactive distillation unit RD as an overhead stream S_{overhead-RD}, and a fraction containing water, which leaves the reactive distillation unit RD as a bottom stream S_{bottom-RD}, takes place.

The methanol-containing stream S_{Feed-RD} has, for example, a methanol concentration C₁(MeOH) of at least 40 mol %, a water concentration C₁(H₂O) of at most 60 mol % and a total concentration of other components (i.e. components that are not methanol and water), if present, of at most 5 mol %.

As mentioned above, the process according to the invention enables efficient synthesis of dimethyl ether even if the methanol-containing stream S_{Feed-RD} used as starting material has a significant amount of water. In a preferred embodiment, therefore, the methanol-containing stream S_{Feed-RD} has a water concentration C₁(H₂O) of 15-60 mol %, more preferably 25-50 mol %, and optionally contains components other than methanol and water in a total concentration of at most 5 mol %.

The methanol-containing stream S_{Feed-RD} may originate from a methanol synthesis unit in which methanol was produced in a known manner (e.g. from synthesis gas, in particular CO₂-rich synthesis gas).

Reactive distillation units suitable for the conversion of methanol to dimethyl ether and water and the separation of the reaction products by distillation are known to those skilled in the art.

The reactive distillation unit RD (e.g. a reactive distillation column) has one or more reaction zones RZ and one or more distillative separation zones DT. The reaction zone RZ contains one or more acid catalysts, in particular one or more acidic solid catalysts. Suitable acid catalysts for the dehydration reaction of methanol to dimethyl ether and water are known to those skilled in the art. For example, the acid catalyst is an ion exchange resin containing acidic groups, a zeolite, an aluminosilicate, an aluminium oxide or an acidic ionic liquid (which is preferably immobilised on a carrier). In the distillative separation zone or distillative separation zones DT, distillative separation takes place into the DME-containing fraction, which leaves the reactive distillation unit RD as an overhead stream S_{overhead-RD}, and the water-containing fraction, which leaves the reactive distillation unit RD as a bottom stream S_{bottom-RD}. The distillative separation zones DT contain, for example, internals for the distillative separation, in particular trays, tower packings or structured packings, as are generally known to those skilled in the art. The immobilisation of the catalyst in the reaction zone RZ of the reactive distillation unit RD can be carried out in a manner known to those skilled in the art, e.g. as random bulk packing; in the form of catalyst-filled wire mesh beads or as catalyst moulded bodies which are attached to a tray in the reaction zone RZ.

By using suitable internals or packings known to those skilled in the art, on which the acid catalyst is present, the reaction zone RZ itself can already provide sufficient distillative separation of the reaction products from one another. Preferably, however, the reactive distillation unit RD has at least one, more preferably at least two, catalyst-free distillative separation zones DT. For example, a catalyst-free distillative separation zone DT may be present in the reactive distillation column RD above and below the reaction zone RZ.

The reactive distillation unit RD is operated, for example, in such a way that a temperature in the range of 100-180° C. and/or a pressure in the range of 8-20 bar is present in the reaction zone RZ.

Preferably, the methanol-containing stream S_{Feed-RD} is fed in the reaction zone RZ of the reactive distillation unit RD. In principle, however, it is also possible to introduce the methanol-containing stream S_{Feed-RD} above or below the reaction zone RZ, for example in a catalyst-free distillation separation zone.

The highly volatile dimethyl ether leaves the reactive distillation unit RD as an overhead stream S_{overhead-RD}, while water (i.e. the component with the highest boiling point) leaves the reactive distillation unit RD as a bottom stream S_{bottom-RD}.

The overhead stream S_{overhead-RD} may have a dimethyl ether concentration of at least 50 mol %, preferably at least 95 mol %, even more preferably at least 99 mol %.

The bottom stream may have a water concentration of at least 50 mol %, preferably at least 90 mol %, even more preferably at least 99 mol %.

In the process according to the invention, a methanol-containing side stream S_{side-RD} is removed from the reactive distillation unit RD (e.g. from the reaction zone RZ of the reactive distillation unit RD) and fed into a side reactor SR. In the side reactor SR, the methanol is converted to dimethyl ether and water in the presence of an acid catalyst to obtain a product stream S_{product-SR}, which contains dimethyl ether, water and methanol and is removed from the side reactor SR.

In the reactive distillation unit RD, the dimethyl ether accumulates in the column head due to its high volatility and the water as the component with the highest boiling point accumulates in the column bottom, while fractions with a high methanol concentration can be removed as a side stream in the intermediate regions of the column. If the reactive distillation unit RD is fed with crude methanol, these fractions that can be removed as a side stream may even have a higher methanol concentration (and therefore also a lower water concentration) than the crude methanol.

The methanol-containing side stream S_{side-RD} removed from the reactive distillation unit RD may have a water concentration C₂(H₂O) of at most 25 mol %, preferably at most 10 mol %, even more preferably at most 5 mol %. Dimethyl ether and, if present, components that are not methanol, water and dimethyl ether, might be present in the methanol-containing side stream S_{side-RD} in a total concentration of at most 10 mol %.

A suitable position or height in the reactive distillation unit RD, at which a side stream with a high methanol concentration or low water concentration can be removed, can be determined by a person skilled in the art on the basis of common general knowledge. For example, the methanol-containing side stream S_{side-RD} is removed at a position relatively high up in the reaction zone RZ, for example in the upper third or upper quarter of the reaction zone RZ. Thus, if the reaction zone RZ has an upper end (i.e. facing the head of the reactive distillation unit RD) and a lower end (i.e. facing the bottom of the reactive distillation unit RD) and a length L (i.e. distance between the upper and lower ends of the reaction zone RZ) and the methanol-containing side stream S_{side-RD} is removed from the reaction zone RZ at a position P_{S}, the position P_{S} may, for example, have a distance I from the upper end of the reaction zone RZ such that I/L≤0.33, more preferably I/L≤0.25.

As already mentioned above, the process according to the invention also enables very efficient preparation of the dimethyl ether if a methanol with a high water content (crude methanol) is used as the starting material. In the context of the present invention, the reactive distillation unit RD is used not only for the synthesis and distillative separation of the dimethyl ether, but also for the provision of a methanol source which has a lower water concentration compared to the crude methanol and thus enables a higher DME yield (compared to crude methanol) in a downstream DME synthesis reactor.

According to an exemplary embodiment, the methanol-containing stream S_{Feed-RD} fed into the reactive distillation unit RD has a water concentration C₁(H₂O) of 15-60 mol %, more preferably 25-50 mol %, and the methanol-containing side stream S_{side-RD} removed from the reactive distillation unit RD has a water concentration C₂(H₂O) which satisfies the following condition:

$${C}_{2} ( {H}_{2} O ) \leq 0.75 \times {C}_{1} ( {H}_{2} O ) .$$

Preferably the following condition applies:

$${C}_{2} ( {H}_{2} O ) \leq 0.6 \times {C}_{1} ( {H}_{2} O ) .$$

For example, the methanol-containing stream S_{Feed-RD} fed into the reactive distillation unit RD may have a water concentration C₁(H₂O) of 15-60 mol %, more preferably 25-50 mol %, and the side stream S_{side-RD} removed from the reactive distillation unit RD may have a water concentration C₂ (H₂O) of at most 10 mol %.

As already mentioned above, the methanol-containing stream S_{Feed-RD} fed into the reactive distillation unit RD may contain components other than methanol and water, in a total concentration of at most 5 mol %. As also mentioned above, the methanol-containing side stream S_{side-RD} removed from the reactive distillation unit RD may contain dimethyl ether and components other than methanol, water and dimethyl ether, in a total concentration of at most 10 mol %.

The side reactor SR can be a reactor type that is usually used for DME synthesis. For example, the side reactor SR is a fixed bed reactor.

For the conversion of methanol to dimethyl ether and water, the side reactor SR contains one or more acid catalysts, in particular one or more acidic solid catalysts. Suitable acid catalysts for the dehydration reaction of methanol to dimethyl ether and water are known to those skilled in the art. For example, the acid catalyst is an ion exchange resin containing acidic groups, a zeolite, an aluminosilicate, an aluminium oxide or an acidic ionic liquid (which is preferably immobilised on a carrier).

In order to work energy-efficiently, it can be advantageous if the side reactor SR is operated at a pressure and a temperature at which the methanol-containing side stream S_{side-RD} fed in and the product stream S_{product-SR} obtained are at least partially present as a liquid phase.

For example, the side reactor SR is operated at a temperature in the range of 130-200° C.

The side reactor SR is preferably operated isothermally. Isothermal operation occurs when the temperature of the reactor in the region of the acid catalyst fluctuates by a maximum of +/−10° C., preferably +/−5° C.

The side reactor SR might be operated at a pressure of 20-150 bar.

The product stream S_{product-SR} of the side reactor SR contains, for example, methanol in a concentration of not more than 50 mol %, preferably not more than 40 mol %. The molar ratio of dimethyl ether to water in the product stream S_{product-SR} is, for example, in the range from 4:6 to 6:4. If present, components that are not dimethyl ether, water and methanol might be present in the product stream S_{product-SR}, for example, in a total concentration of at most 5 mol %.

The methanol contained in the product stream S_{product-SR} can, for example, be at least partially returned to the reactive distillation unit RD as a reactant. In an exemplary embodiment, at least 20 mol %, preferably at least 50 mol %, of the methanol contained in the product stream S_{product-SR} is returned to the reactive distillation unit RD.

In an exemplary embodiment, the product stream S_{product-SR} removed from the side reactor SR and containing dimethyl ether, water and methanol is returned into the reactive distillation unit RD. For example, the product stream S_{product-SR} is returned into the reaction zone RZ of the reactive distillation unit RD.

For example, the product stream S_{product-SR} removed from the side reactor SR might be returned directly to the reactive distillation unit RD. In the context of the present invention, however, it is also possible for the product stream S_{product-SR} removed from the side reactor SR to be divided into at least two partial streams and for at least one of these partial streams to be returned to the reactive distillation unit RD. For example, all partial streams can be returned to the reactive distillation unit. Alternatively, it is also possible that at least one of the partial streams is not returned, for example because it contains dimethyl ether (i.e. the desired product of the process) in high concentration or because it contains undesirable by-products which can be removed from the process in this way.

In an exemplary embodiment, the product stream S_{product-SR} removed from the side reactor SR is divided into three or more partial streams, wherein one of these partial streams has a dimethyl ether concentration of at least 98 mol % or even consists of dimethyl ether and this dimethyl ether is not returned to the reactor distillation unit RD (since it is the desired product of the process). The partial stream consisting substantially of dimethyl ether is obtained, for example, via distillative separation (e.g. from the product stream S_{product-SR}). Alternatively, one of the partial streams contains one or more undesired by-products and these by-products are not returned to the reactive distillation unit but are removed from the process. Preferably, at least two of the partial streams are returned to the reactive distillation unit.

In a further exemplary embodiment, the product stream S_{product-SR} removed from the side reactor SR is fed into a gas-liquid separation unit SU. In this gas-liquid separation unit SU, the product stream S_{product-SR} is separated into a gaseous stream S_{G}, which contains dimethyl ether and methanol (e.g. in a total concentration of at least 80 mol %), and a liquid stream S_{L}, which contains water and methanol (e.g. in a total concentration of at least 80 mol %). For the separation, the product stream S_{product-SR} might be subjected to a pressure reduction, for example. The gaseous stream S_{G} and the liquid stream S_{L} are fed back separately into the reactive distillation unit RD, preferably into the reaction zone of the reactive distillation unit RD.

By separating the product stream S_{product-SR} removed from the side reactor SR into a gaseous stream S_{G} and a liquid stream S_{L} and returning these two streams S_{G} and S_{L} separately to the reactive distillation unit RD, a further improvement in the conversion of methanol to dimethyl ether can be realised compared to the direct return of the product stream S_{product-SR}.

In the reactive distillation unit RD, the reaction zone RZ has a high water concentration and low DME concentration in its lower region. The water concentration decreases towards the head of the reactive distillation unit, so that the upper region of the reaction zone has a very low water concentration. The product stream S_{product-SR} removed from the side reactor SR contains dimethyl ether and water (i.e. the reaction products of the methanol hydration reaction taking place in the side reactor SR) in relatively high concentrations. If, for example, the product stream S_{product-SR} is fed back directly into the upper (i.e. very low water content) region of the reaction zone RZ, this leads to an increase in the water concentration and thus inhibition of the hydration reaction of the methanol in this region of the reaction zone RZ of the reactive distillation unit RD. Alternatively, if the product stream S_{product-SR} is returned, for example, to a lower region (i.e. containing a lot of water but very little DME) of the reaction zone RZ, the increased concentration of the low-boiling component (i.e. dimethyl ether) leads to a lower temperature and thus a lower conversion of the hydration reaction of the methanol in this region of the reaction zone RZ of the reactive distillation unit RD. By separating the product stream S_{product-SR} removed from the side reactor SR into a gaseous stream S_{G}, which contains predominantly DME and methanol but little water, and a liquid stream S_{L}, which contains predominantly water and methanol but little DME, and returning these two streams S_{G} and S_{L} separately into the reactive distillation unit RD at suitable positions, a further improvement in the conversion of methanol to dimethyl ether can thus be realised compared to a direct recirculation of the product stream S_{product-SR}.

Preferably, the gaseous stream S_{G} is fed into the reactive distillation unit RD (e.g. the reaction zone RZ) at a position P1 and the liquid stream S_{L} at a position P2, so that the position P1 is above the position P2. The term “above” means that position P1 is closer to the head of the distillation unit than position P2.

For example, the position P1 is located in the upper third (preferably in the upper fifth) of the reaction zone RZ or in a catalyst-free distillative separation zone DT located above the reaction zone RZ, and the position P2 is located in the lower half of the reaction zone RZ or in a catalyst-free distillative separation zone DT located below the reaction zone RZ.

A position P1 in the upper third of the reaction zone means the following: The reaction zone RZ has an upper end (i.e. facing the head of the reactive distillation unit RD) and a lower end (i.e. facing the bottom of the reactive distillation unit RD) and a length L (i.e. distance between the upper and lower ends of the reaction zone RZ) and the position P1 has a distance I₁ from the upper end of the reaction zone RZ, so that I₁/L≤0.33. For a position P1 in the upper fifth of the reaction zone RZ, the following applies: I₁/L≤0.2.

A position P2 in the lower half of the reaction zone means the following: The reaction zone RZ has an upper end (i.e. facing the head of the reactive distillation unit RD) and a lower end (i.e. facing the bottom of the reactive distillation unit RD) and a length L (i.e. distance between the upper and lower ends of the reaction zone RZ), and the position P2 has a distance I₂ from the lower end of the reaction zone RZ such that I₂/L≤0.5.

Suitable gas-liquid separation units for separation into a gas phase and a liquid phase are known to those skilled in the art. For example, the separation involves subjecting the product stream S_{product-SR} to a pressure reduction in a container so that a gaseous phase containing dimethyl ether and methanol and a liquid phase containing water and methanol are formed and the gaseous and liquid phases are separated from each other. The gas-liquid separation unit SU is, for example, a flash separator.

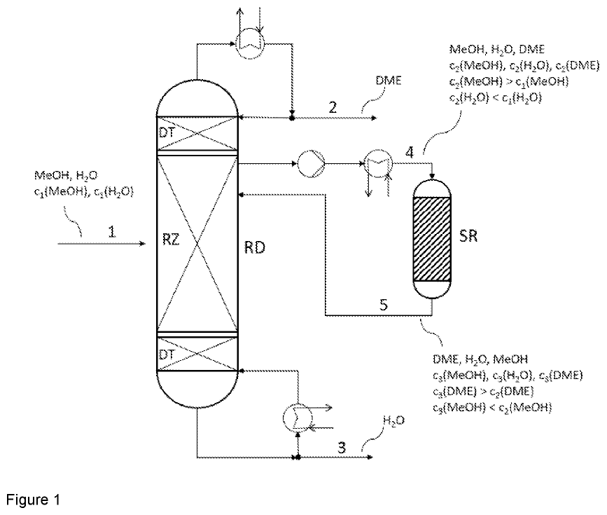

An exemplary embodiment of the present invention is described in greater detail with reference to FIG. 1:

A methanol-containing stream S_{Feed-RD} is fed into the reaction zone RZ of a reactive distillation column RD via line 1. The methanol-containing stream S_{Feed-RD} is, for example, crude methanol that, in addition to MeOH, contains a significant proportion of H₂O (c₁(MeOH):methanol concentration; c₁(H₂O):water concentration). If the reaction zone RZ already has a sufficient distillative separation effect due to the internals used, the presence of catalyst-free distillative separation zones can be dispensed with. In the embodiment illustrated in FIG. 1, there is a catalyst-free distillative separation zone DT above and below the reaction zone RZ.

In the reactive distillation unit RD, methanol is converted to dimethyl ether and water in the presence of an acid catalyst and is separated by distillation into a fraction which contains dimethyl ether and leaves the reactive distillation unit RD via a line as an overhead stream S_{overhead-RD}, and a fraction which contains water and leaves the reactive distillation unit RD via line 3 as a bottom stream S_{bottom-RD}. The overhead stream S_{overhead-RD} substantially contains dimethyl ether (e.g. in a concentration of at least 99 mol %) and the bottom stream substantially contains water (e.g. in a concentration of at least 99 mol %).

A methanol-containing side stream S_{side-RD} is removed from the reactive distillation unit RD via line 4 and fed to a side reactor SR. The side stream S_{side-RD} contains mainly methanol and small amounts of water and dimethyl ether (c₂(MeOH):methanol concentration; c₂(H₂O):water concentration; c₂(DME): dimethyl ether concentration). The removed methanol-containing side stream S_{side-RD} has a higher methanol concentration and lower water concentration (i.e. c₂(MeOH)>c₁(MeOH); c₂(H₂O)<c₁(H₂O)) compared to the stream S_{Feed-RD} fed into the reactive distillation unit RD.

The side reactor SR is operated isothermally (e.g. at a temperature in the range of 130-200° C.) and contains an acid catalyst for the dehydration reaction of methanol to dimethyl ether and water. In order to work energy-efficiently, the side reactor SR is operated in such a way that the methanol-containing side stream S_{side-RD} fed in and the product stream S_{product-SR} obtained in the side reactor SR are not completely in the gas phase, but at least partially in the liquid phase.

The product stream S_{product-SR} containing dimethyl ether, water and methanol is removed from the side reactor SR via line 5 (c₃(DME): dimethyl ether concentration; c₃(H₂O):water concentration; c₃(MeOH):methanol concentration) and returned to the reaction zone RZ of the reactive distillation unit RD. Since methanol is converted to dimethyl ether (and water) in the side reactor SR, the product stream S_{product-SR} has a higher dimethyl ether concentration and lower methanol concentration compared to the side stream S_{side-RD} (c₃(DME)>c₂(DME); c₃ (MeOH)<c₂(MeOH)).

The reactive distillation unit RD is not only used for the synthesis and distillative separation of the dimethyl ether, but also in the form of the methanol-containing side stream S_{side-RD} removed via line 4 for the provision of a methanol source, which has a lower water concentration compared to the crude methanol fed to the reactive distillation unit RD via line 1 and thus enables a higher DME yield (compared to crude methanol) in the product stream S_{product-SR} in the downstream-located side reactor. The recirculation of the DME-rich product stream S_{product-SR} to the reactive distillation unit RD in turn has a favourable influence on the yield of dimethyl ether in the reactive distillation unit RD.

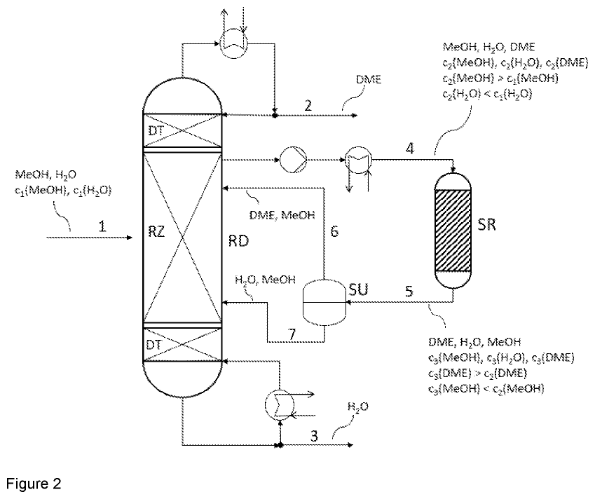

A further exemplary embodiment of the process according to the invention is described in greater detail with reference to FIG. 2. The process management illustrated in FIG. 2 differs from the process management illustrated in FIG. 1 as follows:

The product stream S_{product-SR} removed from the side reactor SR via line 5 is fed into a gas-liquid separation unit SU. In this gas-liquid separation unit SU, the product stream S_{product-SR} is separated into a gaseous stream S_{G}, which predominantly contains dimethyl ether and methanol (e.g. in a total concentration of at least 80 mol %), and a liquid stream S_{L}, which predominantly contains water and methanol (e.g. in a total concentration of at least 80 mol %). The gaseous stream S_{G} is returned to the reactive distillation unit RD via line 6 and the liquid stream S_{L} is returned to the reactive distillation unit RD via line 7.

As already described above, by separating the product stream S_{product-SR} removed from the side reactor SR into a gaseous stream S_{G} and a liquid stream S_{L} and returning these two streams S_{G} and S_{L} separately to the reactive distillation unit RD, a further improvement in the conversion of methanol to dimethyl ether can be realised compared to a direct return of the product stream S_{product-SR}.

With regard to all other features of the exemplary embodiment illustrated in FIG. 2, reference can be made to the above description of FIG. 1.

1. A process for the preparation of dimethyl ether, comprising:
feeding a methanol-containing stream S_{Feed-RD} into a reactive distillation unit RD, wherein
in at least one reaction zone RZ of the reactive distillation unit RD, methanol is converted to dimethyl ether and water in the presence of an acid catalyst, and
a distillative separation is performed into a fraction that contains dimethyl ether and leaves the reactive distillation unit RD as an overhead stream S_{overhead-RD}, and a fraction that contains water and leaves the reactive distillation unit RD as a bottom stream S_{bottom-RD},
removing a methanol-containing side stream S_{side-RD} from the reactive distillation unit RD,
feeding the methanol-containing side stream S_{side-RD} into a side reactor SR and converting the methanol in the presence of an acid catalyst to dimethyl ether and water to obtain a product stream S_{product-SR}, which contains dimethyl ether, water and methanol and is removed from the side reactor SR.

2. The process according to claim 1, wherein the methanol-containing stream S_{Feed-RD} fed into the reactive distillation unit RD has a methanol concentration C₁(MeOH) of at least 40 mol %, a water concentration C₁(H₂O) of at most 60 mol % and a total concentration of other components which are not methanol and water of at most 5 mol %.

3. The process according to claim 1, wherein the methanol-containing stream S_{Feed-RD} fed into the reactive distillation unit RD has a water concentration C₁(H₂O) of 15-60 mol %, and the methanol-containing side stream S_{side-RD} removed from the reactive distillation unit RD has a water concentration C₂(H₂O) which satisfies the following condition:
$$[ [ c ] ] {\underline{C}}_{2} ( {H}_{2} O ) \leq 0.75 \times [ [ c ] ] {\underline{C}}_{1} ( {H}_{2} O ) .$$

4. The process according to claim 1, wherein the methanol contained in the product stream S_{product-SR} is at least partially returned to the reactive distillation unit RD.

5. The process according to claim 1, wherein the product stream S_{product-SR} removed from the side reactor SR is returned to the reactive distillation unit RD.

6. The process according to claim 1, further comprising:
feeding the product stream S_{product-SR} removed from the side reactor SR into a gas-liquid separation unit SU and separating the product stream S_{product-SR} into a gaseous stream S_{G}, which contains dimethyl ether and methanol, and a liquid stream S_{L}, which contains water and methanol,
separately returning the gaseous stream S_{G} and the liquid stream S_{L} to the reactive distillation unit RD.

7. The process according to claim 6, wherein the gaseous stream S_{G} is fed into the reactive distillation unit RD at a position P1 and the liquid stream S_{L} is fed into the reactive distillation unit RD at a position P2 and the position P1 is located in the upper third of the reaction zone RZ or in a catalyst-free distillative separation zone DT located above the reaction zone RZ and the position P2 is located in the lower half of the reaction zone RZ or in a catalyst-free distillative separation zone DT located below the reaction zone RZ.
